(19) United States
(12) Patent Application Publication
(10) Pub. No.: US 2025/0238925 A1
(43) Pub. Date: Jul. 24, 2025

(54) Classification of Cognitively Normal Condition, Mild Cognitive Impairment and Alzheimer's Disease Based on Convolutional Neural Networks with Attention Mechanism

(71) Applicant: City University of Hong Kong, Hong Kong (CN)

(72) Inventors: Kei Hang Katie CHAN, Hong Kong (CN); Yat Ming Peter WOO, Hong Kong (CN); Jundong LIU, Hong Kong (CN); Mingwei YAO, Hong Kong (CN); Jingtao CHEN, Hong Kong (CN)

(21) Appl. No.: 19/032,524

(22) Filed: Jan. 21, 2025

Related U.S. Application Data
(60) Provisional application No. 63/622,738, filed on Jan. 19, 2024.

(51) Int. Cl. G06T 7/00; A61B 5/00; A61B 5/055; G06V 10/764; G06V 10/82; G16H 50/30

(52) U.S. Cl. CPC G06T 7/0012; A61B 5/0042; A61B 5/055; A61B 5/4088; G06V 10/764; G06V 10/82; G16H 50/30; G06T 2207/10088; G06T 2207/20081; G06T 2207/20084; G06T 2207/30008; G06V 2201/031

(57) ABSTRACT

An image processing framework for multi-class classifying a subject into cognitive normal, mild cognitive impairment and Alzheimer's disease (AD) conditions is developed. In one realization of the framework, an AD_Net model, which is an attention-enhanced convolution neural network (CNN) formed by embedding a Convolutional Block Attention Module (CBAM) into a CNN having a Visual Geometry Group 19 (VGG19) architecture, processes an image volume of the subject's brain to generate a plurality of AD_Net feature maps and a first plurality of scores that predict respective likelihoods of the three conditions. To enhance the prediction accuracy, a multilayer perception model formed with a plurality of fully connected layers processes the plurality of AD_Net feature maps and a plurality of influencing factors of AD, such as age, gender, geriatric depression scale score, Mini-Mental State Examination score and clinical dementia rating score, to generate a second plurality of scores that predict the respective likelihoods.

### CROSS-REFERENCE TO RELATED APPLICATIONS

This application claims priority to, and the benefit of, U.S. Provisional Patent Application No. 63/622,738 filed on Jan. 19, 2024, the disclosure of which is hereby incorporated by reference in its entirety.

### ABBREVIATIONS

3D three-dimensional
AD Alzheimer's disease
ADNI Alzheimer's Disease Neuroimaging Initiative
AGG age, gender, GDS
AGMGC age, gender, MMSE, GDS, CDR
AIBL Australian Imaging, Biomarkers and Lifestyle
APOE apolipoprotein E
AUROC area under the ROC curve
CAD computer-aided diagnosis
CBAM Convolutional Block Attention Module
CDR clinical dementia rating
CN cognitively normal
CNN convolutional neural network
FMRIB Functional Magnetic Resonance Imaging of the Brain
FSL FMRIB Software Library
GDS geriatric depression scale
MCI mild cognitive impairment
ML machine learning
MLP multilayer perceptron
MMSE Mini-Mental State Examination
MRI magnetic resonance imaging
NC normal control
ROC receiver operating characteristic
TPF true positive fraction
VGG Visual Geometry Group

### TECHNICAL FIELD

The present disclosure relates to using a ML model with an attention mechanism to classify a subject into a CN condition, a MCI condition and an AD condition.

### BACKGROUND

AD is a neurodegenerative disease with insidious onset and progressive development. AD is characterized by an age-related increase in prevalence. Between the ages of 65 and 85, the chance of prevalence increases nearly 15-fold, according to studies [1]. Although there is currently no effective treatment for AD, early diagnosis is necessary to help people prevent development of this disease or slow down progression thereof. Early disease detection is critical for early treatment.

Clinically, AD is characterized by the accumulation of extracellular amyloid plaques, intracellular neurofibrillary tangles, and loss of neurons and synapses. These changes result in brain atrophy [2]. Therefore, to further study AD, it is necessary to analyze the change in brain structure through MRI. Biological institutions have already researched AD and collected useful data. Such studies include the AIBL Study and the ADNI, both of which produced comprehensively coordinated data sets [3].

The CAD Dementia challenge was a challenge with standard protocols and hidden test labels to evaluate and compare different AD prediction methods [4]. The winning scheme [5] realized the classification of CN, MCI, and AD, and achieved a remarkable accuracy of 63%. However, the accuracy was boosted only by calculating the shape of hippocampus, which required pre-calculation of the test set, human intervention, and up to 19 hours of analysis for each subject.

Currently, many ML-based studies of AD cases use the CNN framework [6], [7]. However, few studies have used 3D data input to classify and predict the conditions of CN, MCI, and AD. Moreover, in recent years, attention mechanisms like CBAM have gained widespread application in computer vision and natural language processing, significantly enhancing the capability of CNN models to extract more valuable feature maps. However, integrating CNNs with attention mechanisms with the purpose of using the 3D data input to classify and predict CN, MCI and AD has largely been unexplored. There is a need in the art to explore a technique that integrates CNNs with attention mechanisms for the aforementioned purpose.

### SUMMARY

An aspect of the present disclosure is to provide a computer-implemented method for classifying a subject into a CN condition, a MCI condition and an AD condition.

The method comprises: obtaining an image volume of the subject's brain; and using an AD_Net model to process the image volume to thereby generate a plurality of AD_Net feature maps and a first plurality of scores that respectively predict likelihoods of the CN, MCI and AD conditions. In particular, the AD_Net model is an attention-enhanced CNN formed by embedding an attention module into a CNN module.

In certain embodiments, the method further comprises classifying the subject into the CN, MCI and AD conditions according to the first plurality of scores.

In certain embodiments, the method further comprises training the AD_Net model before the AD_Net model is used to process the image volume.

Preferably and advantageously, the method further comprises: obtaining a plurality of non-directionally influencing factors of AD, and a plurality of directionally influencing factors of AD; using a MLP model to process an input to thereby generate a second plurality of scores that respectively predict the likelihoods of the CN, MCI and AD conditions, wherein the input includes the plurality of non-directionally influencing factors of AD, the plurality of directionally influencing factors of AD, and the plurality of AD_Net feature maps, and wherein the MLP model fuses the feature maps with the non-directionally and directionally influencing factors in at least one feature-fusion layer; and classifying the subject into the CN, MCI and AD conditions according to the second plurality of scores.

In certain embodiments, the plurality of non-directionally influencing factors of AD includes one or more first items selected from an age, a gender and a GDS score.

In certain embodiments, the plurality of directionally influencing factors of AD includes one or more second items selected from a MMSE score and a CDR score.

In certain embodiments, the MLP model comprises a plurality of fully connected layers. Furthermore, LeakyReLU is used as an activation function in the plurality of fully connected layers.

In certain embodiments, the method further comprises training the AD_Net model and MLP model before the AD_Net model is used to process the image volume.

In certain embodiments, the CNN module is realized as a VGG model and the attention module is realized as a CBAM.

In certain embodiments, the VGG model is an optimized VGG19 model.

In certain embodiments, the CBAM is added to the VGG19 model at a location after a final 64-channel convolution layer of the VGG19 model.

In certain embodiments, the image volume is prepared from data obtained from three-dimensionally imaging the subject's brain by MRI.

In certain embodiments, the method further comprises: obtaining a raw-image volume of the subject's brain; and preprocessing the raw-image volume to generate the image volume such that the image volume is obtained.

In certain embodiments, the raw-image volume is pre-processed by performing linear registration, skull removal, bias field correction, and noise cutting and normalization.

A computing system for classifying a subject into CN, MCI and AD conditions is realizable based on the disclosed method. In particular, the computing system comprises one or more computers configured to execute a process of classifying the subject into the CN, MCI and AD conditions according to any of the embodiments of the disclosed method.

Other aspects of the present disclosure are disclosed as illustrated by the embodiments hereinafter.

### BRIEF DESCRIPTION OF THE DRAWINGS

FIG. 1 depicts a schematic diagram of an exemplary ML-based image processing framework as disclosed herein for classifying a subject into CD, MCI and AD conditions, where the framework uses: an AD_Net model, which is an attention-enhanced CNN, to process an image volume of a subject's brain to generate a plurality of AD_Net feature maps; a MLP model to process the AD_Net feature maps and preselected influencing factors of AD to generate a plurality of scores indicating respective likelihoods of the above-mentioned three conditions; and data preprocessing operation(s) to obtain the image volume from a raw-image volume.

FIG. 2 depicts a schematic diagram illustrating a structure of a spatial attention module used in a CBAM, where the CBAM provides an attention mechanism to the AD_Net model.

FIG. 3 depicts a schematic diagram illustrating a structure of a channel attention module used in a CBAM.

FIG. 4 depicts a schematic diagram showing a structure of the CBAM.

FIG. 5 depicts a schematic structure of each convolution block as used in the AD_Net model.

FIG. 6 depicts structures of convolution blocks and VGG19 after directional optimization.

FIG. 7 depicts a schematic structure of the AD_Net model.

FIG. 8 illustrates compositions of external influencing factors, where: a group of AGG influencing factors includes age, gender and GDS score; and a group of AGMGC influencing factors includes age, gender, MMSE score, GDS score and CDR score.

FIG. 9 depicts a schematic structure of the MLP model.

FIG. 10 plots ROC curves experimentally obtained in AD_Net tests.

FIG. 11 plots ROC curves experimentally obtained in ADNet_AGG tests.

FIG. 12 plots ROC curves experimentally obtained in ADNet_AGMGC tests.

Skilled artisans will appreciate that elements in the figures are illustrated for simplicity and clarity and have not necessarily been depicted to scale.

### DETAILED DESCRIPTION

The present disclosure discloses a ML-based image processing framework, which integrates CNNs with attention mechanisms, for multi-class classification of a subject into CD, MCI and AD conditions. The image processing framework is developed based on a ML-based AD-prediction model, herein referred to as the AD_Net model, our AD_Net model, our AD_Net, or AD_Net in short. After the ML-based image processing framework is detailed, embodiments of the present disclosure will be elaborated based on the disclosed details, examples, applications, etc. of the framework.

### I. Overview of the Image Processing Framework

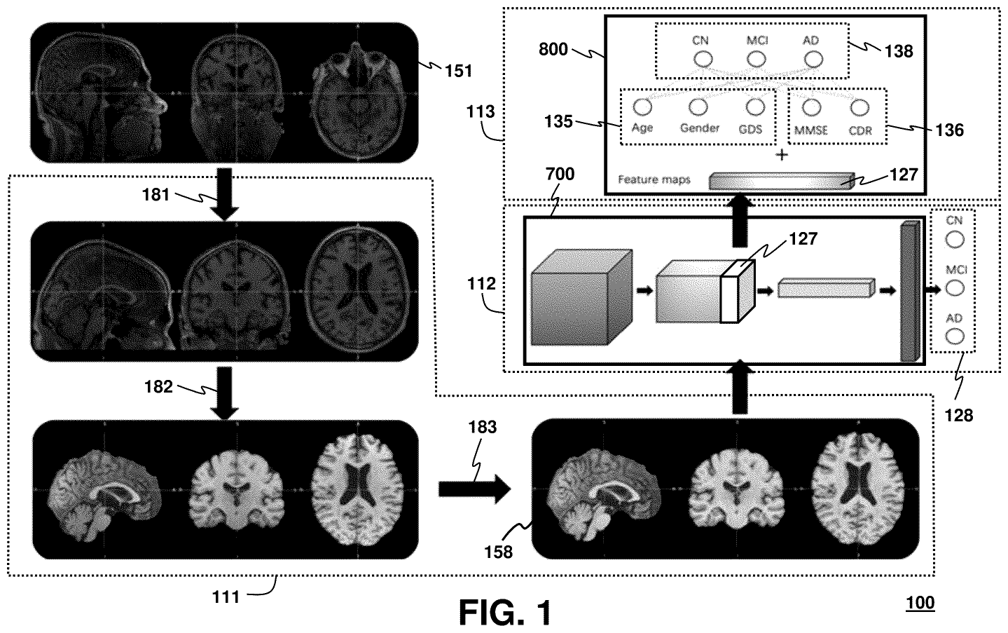

The disclosed framework is exemplarily illustrated with the aid of FIG. 1, which depicts an exemplary ML-based image processing framework 100 for classifying a subject into CD, MCI and AD conditions.

In the framework 100, an AD_Net model 700 is used to process an image volume 158 of the subject's brain to thereby generate a plurality of AD_Net feature maps 127 and a first plurality of scores 128 that respectively predict likelihoods of the CN, MCI and AD conditions. In particular, the AD_Net model 700 is an attention-enhanced CNN. Usually, the image volume 158 is obtained by first obtaining a raw-image volume 151 of the subject's brain (e.g., through MRI) and then performing a data preprocessing operation 111 on the raw-image volume 151 to obtain the image volume 158. The classification of the subject into the CD, MCI and AD conditions may be determined according to the first plurality of scores 128.

The AD_Net model 700 advantageously uses CBAM to implement the attention mechanism. Our selection of CBAM has notably improved the prediction performance and stability of our AD_Net 700, making it more suitable for actual disease diagnosis. CBAM's effectiveness extends across a broad spectrum of medical imaging tasks. For example, it has been used to enhance electrocardiogram feature maps for diagnosing heart failure [8], aid in the segmentation of brain tumors, and even detect schizophrenia using deep learning networks with patient speech as input [9]. Additionally, CBAM has been proven beneficial in diagnosing a variety of pathological brain diseases, including cerebrovascular, neoplastic, degenerative, and infectious diseases [6], [9]-[13]. The combination of VGG19 and CBAM is chosen as the basis for our AD_Net model 700.

Apart from the image volume 158, which contains dimensional information of anatomical features, such as a hippocampus, of the subject's brain, additional influencing factors of AD may be taken into consideration in classifying the subject into the CD, MCI and AD conditions. According to the diagnosis of the disease, a MLP model 800 may be used in conjunction with the AD_Net model 700 to embed potential influencing factors both non-directionally and directionally. Specifically, it is preferable and advantageous that the MLP model 800 is used in the framework 100 to process an input to thereby generate a second plurality of scores 138 that respectively predict likelihoods of the CN, MCI and AD conditions, where the input consists of a plurality of non-directionally influencing factors 135 of AD, a plurality of directionally influencing factors 136 of AD, and the plurality of AD_Net feature maps 127. The non-directionally influencing factors 135 may include factors such as age, gender, and GDS [14], whilst the directionally ones 136 may include MMSE score [15] and CDR [16]. Potential influencing factors with broader implications may also be used. The subject is classified into the CN, MCI and AD conditions according to the second plurality of scores 138.

### II. Framework Development: Materials and Method

As a representative configuration of the framework 100 for illustrating the framework development without loss of generality, the framework 100 under consideration performs a data preprocessing operation 111, a 3D CNN processing operation 112 and a MLP prediction operation 113. The data preprocessing operation 111 is arranged to perform linear registration 181, skull removal 182, and various signal-conditioning operations 183 including bias field correction, and normalization and noise cutting.

### A. Data Source

All the raw data we used in the development and testing of the image processing framework 100 were MRI data obtained from the ADNI's open-source database [17]. There were 437 subjects in our study, and all MRI data were collected when the subject was first recruited. We selected CN, MCI, and AD as the three different disease states, all of which were T1-weighted and MR-RAGE.

Our dataset was multi-class, with each subject represented by a single MRI image. We applied a stratified 5-fold split for the 437 subjects: approximately 65 to 71 CN, 133 to 148 MCI, and 51 to 60 AD cases in each training set. Validation and test sets maintained proportional distributions, typically including around 22 to 27 CN, 36 to 51 MCI, and 14 to 23 AD cases for validation, and consistently 29 CN, 38 MCI, and 21 AD cases for testing.

We dealt with the imbalance issue in the dataset by using the PyTorch sampler to sample data with a probability according to the count of each class so that each mini-batch had the same expectation counts of samples from each class. The data selected by this approach could be directly used for model training, equivalent to regulating data selection.

### B. Data Preprocessing 111

The ultimate objective of the data preprocessing operation 111 is to transform MRI images into a 3D tensor of dimensions 145×182×155. This transformation involves several key steps designed to optimize the data for use with our CNN model, ensuring comprehensive coverage of the brain region while minimizing background noise and other non-relevant signals [18]. It involves the following major steps: linear registration 181; skull removal 182; and various signal-conditioning steps 183, including bias field correction, and noise cutting and normalization.

Linear registration 181: To ensure consistency across different MRI scans, we utilized the FSL tool for linear registration during the development and testing of the framework 100. This process aligned each MRI image to a standard template, specifically the MNI152 T1, ensuring that anatomical locations were consistent across images.

Skull removal 182: We employed the bet2 function from FSL to remove the skull and other non-brain structures from the images. This step was critical as it focused the analysis on brain tissue only, eliminating potential distractions and irrelevant data.

Bias field correction: MRI images often suffer from intensity inhomogeneities due to variations in the magnetic field during the scanning process. These variations can cause parts of the image to appear lighter or darker, affecting the accuracy of subsequent analyses. In the development of the framework 100, we addressed this issue by using the N4BiasFieldCorrectionImageFilter from SimpleITK, which normalized these intensity variations across the entire image.

Noise cutting and normalization: After correcting for any intensity biases, we converted the MRI data from the NIfTI format to NumPy arrays, reshaping it into the desired 3D tensor dimensions of 145×182×155. This specific dimensioning ensured that almost the entire brain was captured with minimal inclusion of surrounding areas. We then normalized the data using the mean and standard deviation calculated from the dataset, adjusting the intensity values to a common scale suitable for neural network processing. This normalization was crucial for effective learning and generalization across different scans. Finally, we applied a clipping function to remove outlier intensity values, setting a range of −1 to 2.5, which helped in further reducing noise and enhancing the ability of the AD_Net model 700 to focus on relevant features.

### C. Performance Metrics

The performance of the framework 100 was evaluated using two primary metrics: accuracy and the AUROC. To ensure the robustness and reliability of these metrics, we employed a five-fold cross-validation method, which was repeated three times.

### D. CBAM

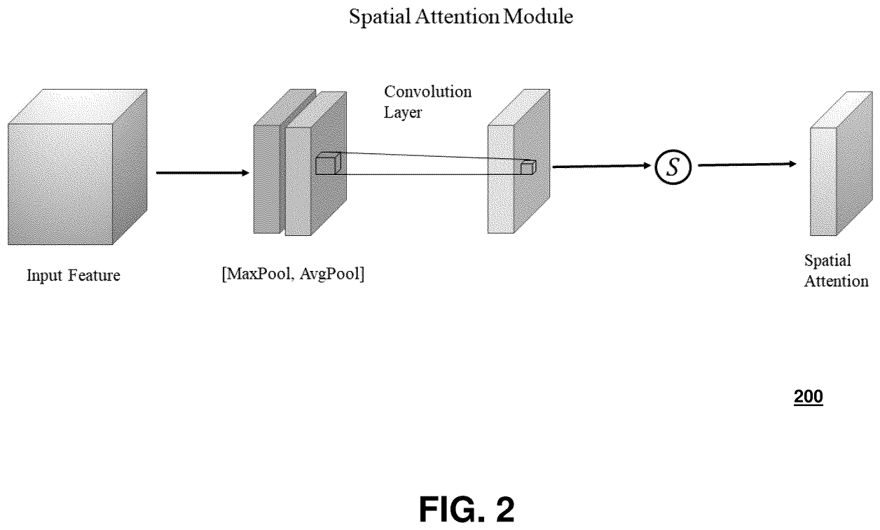

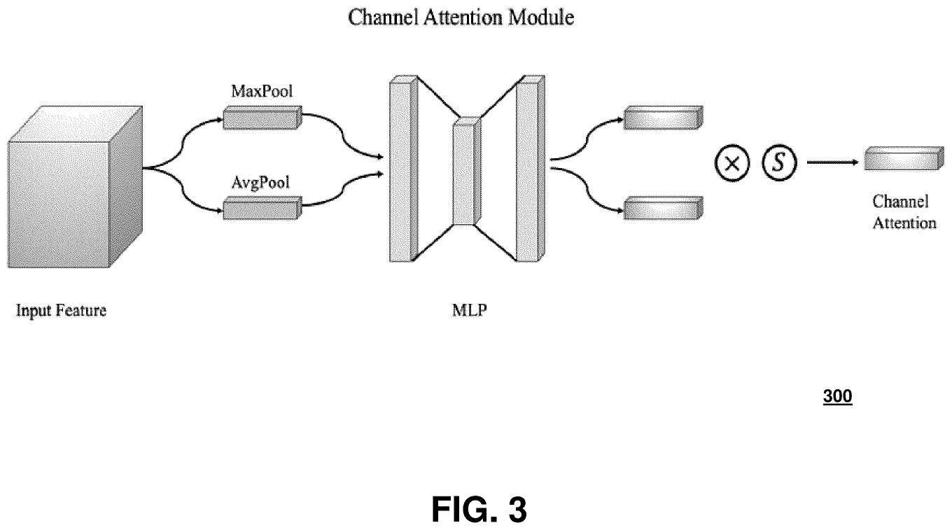

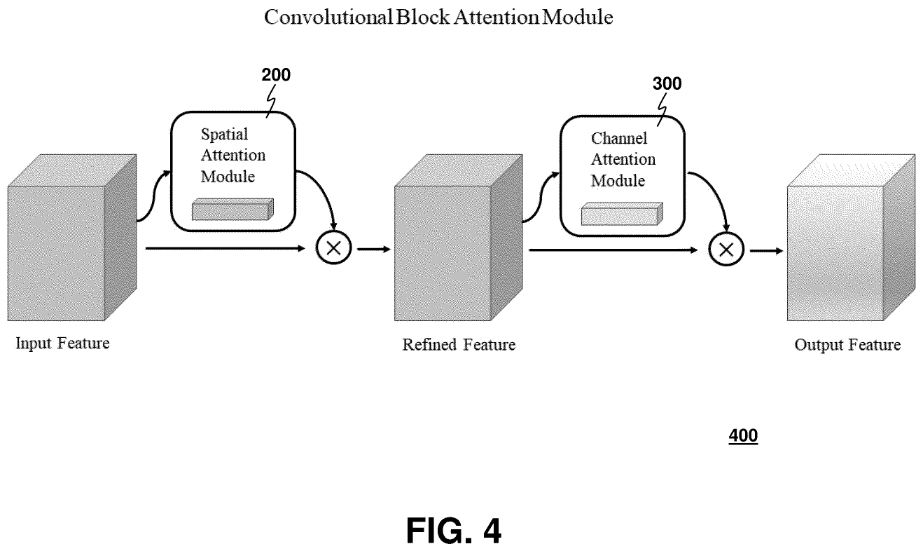

FIG. 4 depicts a schematic diagram of a CBAM 400. The CBAM 400 as used in the present disclosure combines a spatial attention module 200 and a channel attention module 300. Schematic diagrams of the spatial attention module 200 and the channel attenuation module 300 are depicted in FIGS. 2 and 3, respectively.

As shown in FIG. 2, the calculation of spatial attention weight in the spatial attention module 200 first reduces the dimensionality of the input characteristic values to obtain the results of maximum pooling and mean pooling, respectively, and then splices again into a feature map. The generated feature graph is then taken as input and the convolution layer is used to learn the spatial weight.

In contrast, channel attention weight uses MLP to learn the results of maximum pooling and mean pooling, respectively, and then overlays them. The output is processed with a sigmoid activation function to get the channel weight in the module. The structure can be seen in FIG. 3.

The CBAM 400 obtains the spatial weight through the spatial attention module 200 and then multiplies it with the original feature maps to obtain the first set of optimized feature values. These are used as input for channel attention to obtain the channel weight, and finally, the channel weight is multiplied by the results of the first set of refined feature maps to result in the final optimized feature maps.

### E. 3D CNN Processing 112

We established several mainstream CNN architectures for prediction analysis, and the classical architectures with good performance were selected for further improvement to achieve structural optimization for AD prediction. The 3D CNN processing operation 112 is responsible for using the AD_Net model 700 optimized with the best prediction performance to process the image volume 158 and extracting the plurality of AD_Net feature maps 127 generated from a fully connected layer of the AD_Net model 700 as one of the inputs to the MLP model 800.

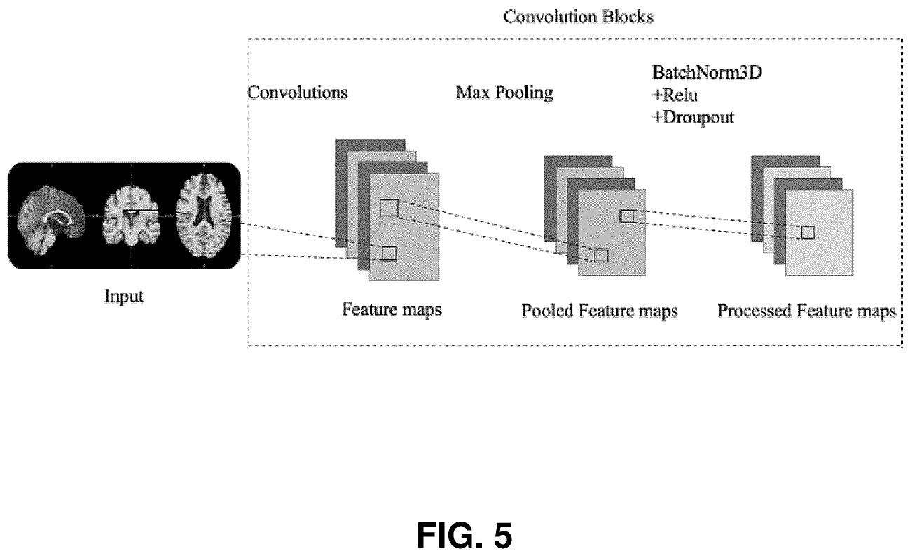

The candidate ML models we considered for developing the AD_Net model 700 included 3D CNN baseline, AlexNet, VGG13, VGG16 and VGG19. AlexNet, with a classic CNN architecture, had an excellent iterative process. The VGG model introduced the concept of convolution block based on AlexNet to build a deeper and larger model. We built four convolution blocks according to VGG architecture to construct the 3D CNN baseline model. FIG. 5 depicts a schematic structure of each convolution block as used in the AD_Net model 700. As shown in FIG. 5, each convolution block was composed of five parts: Convolution layer, Maxpooling layer, BatchNorm, ReLU, and Dropout. The initial input channel was set as 1 and the output channel as 20; these values were doubled as the convolution block was iterated. The convolution kernel size of the first block was set as 7, and the padding size was set as 2. The convolution kernel size was gradually reduced to 3 starting from the third block to build the iterative mode of first broadening and then narrowing. The prediction accuracy of the baseline is a measure of the prediction difficulty levels of the three categories.

We evaluated the prediction performance of each algorithm, including AlexNet, VGG13, VGG16, and VGG19, and made a comparison. We also imported Densnet121 from the MONAI medical image processing library to compare the processing results of known mature models.

After testing all the classical models, we finally chose the VGG model as the basic model for optimization because of its superiority. Considering that the deeper model is more robust, we finally conducted targeted optimization and comparison based on VGG16 and VGG19.

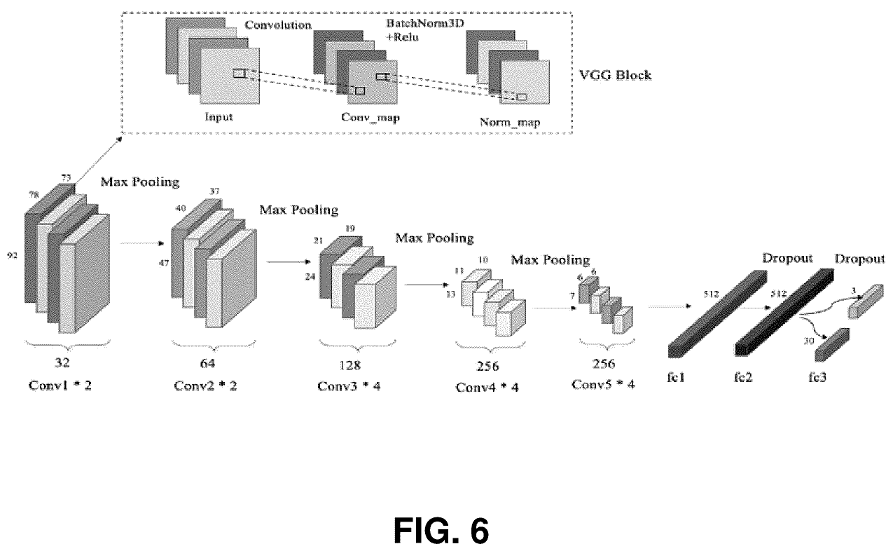

Compared with the classical architecture, we optimized three aspects. First, we reduced the number of files input in each training by half. This reduced the burden of the computing and was more suitable for MRI image analysis. Secondly, we changed the number of nerve nodes in the whole connection layer to 512. Third, we added a dropout layer to each fully connected part. Both optimization two and three can effectively prevent the overfitting problem in the training process. The convolution blocks and structure of VGG19 after directional optimization is shown in FIG. 6.

In the convolution process, the size of the convolution kernel and pooling layer were set to 3 and 2, respectively, and the stride size of both was set to 1.

After establishing the optimization of a pure CNN module, we started to embed the attention module. We added the CBAM 400 after the convolutional layer 64 and concatenated the processed feature maps with the original VGG output together as the input to the fully connected layer. The AD_Net model 700, which is an attention-enhanced CNN, was obtained.

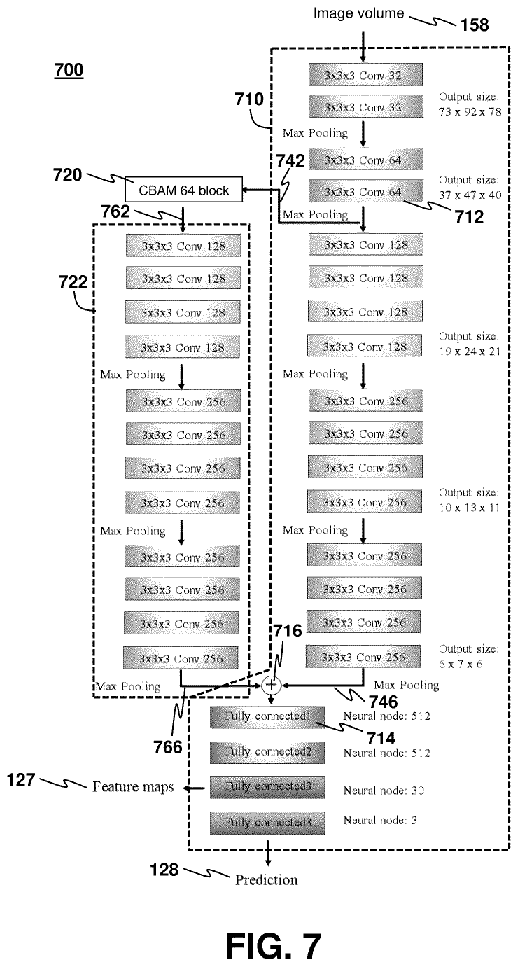

A schematic structure of the AD_Net model 700 is shown in FIG. 7. The AD_Net model 700 as finalized was a fusion of optimized VGG19 and CBAM64.

### F. MLP Processing 113

The MLP model 800 contains a hidden layer with the number of neural nodes set to 8 where we extracted five types of characteristic information from metadata and based on a comprehensive literature review of established biomarkers for AD and consultations with clinical experts, including age, gender, MMSE score, CDR score and GDS score. Initially, we included the APOE allele, a well-documented risk factor for AD [19], but subsequently excluded it due to its lower predictive performance. The GDS score is a widely utilized tool for screening depressive symptoms in the elderly [14]. The MMSE score serves as a prevalent tool for assessing cognitive functions and detecting cognitive impairments and dementia [20]. The CDR provides a multidimensional scoring system to evaluate the severity of dementia [21]. Demographic characteristics, such as age and gender, are typically gathered as baseline data to evaluate their potential influence on cognitive functions and the risk of developing dementia. The literature indicates that older age, higher GDS and CDR scores, and female gender are positively associated with AD, whereas higher MMSE scores may inversely correlate with the condition [20]-[24].

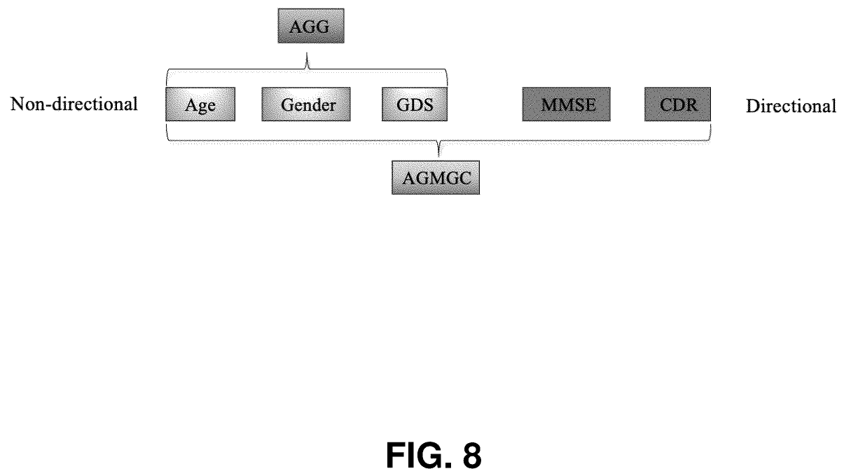

These factors were divided into two groups, with and without the directional influence factors. We called these two groups AGG and AGMGC as shown in FIG. 8, and we took these two groups as two independent inputs and combined them with the plurality of AD_Net feature maps 127 generated by VGG16, VGG19, and our AD_Net 700 to form a total of eight control groups for performance tests.

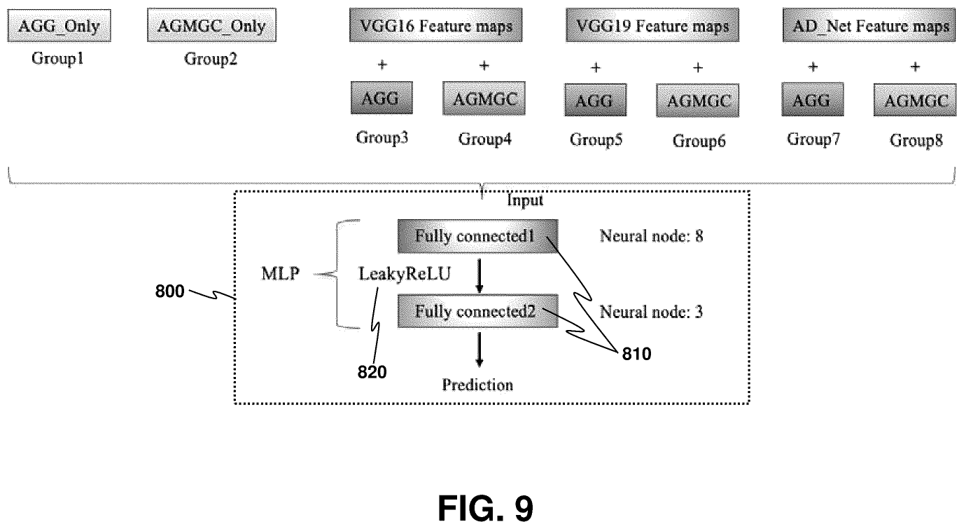

Our MLP model 800 used for conducting the performance tests is shown in FIG. 9. As shown in FIG. 9, our MLP model 800 used LeakyReLU as an activation function and set one hidden layer with several nerve nodes set to 8. In the MLP model 800, we used the same five-fold validation data set as the convolution module, output five groups of training, validation, and test accuracy, and finally took the mean value to evaluate the prediction and classification performance of each group.

### III. Experimental Results

### A. Performance of the AD Net Model 700

As detailed in TABLE I, the accuracy of each of different CNN models under test (including the AD_net model 700) is quantified by both the mean and standard deviation. Our results demonstrate that the optimized AD_Net 700 matched the accuracy of VGG16 but exhibited a smaller standard deviation, highlighting enhanced robustness of the AD_Net model 700. Despite VGG16's commendable performance, our specialized optimizations resulted in a modest prediction accuracy of 48%. In contrast, similar optimizations applied to VGG19 led to a higher prediction accuracy of 50.9%. Most notably, the AD_Net model 700 achieved an accuracy of approximately 52%, marking the highest rate documented in related studies up to October 2020 [18].

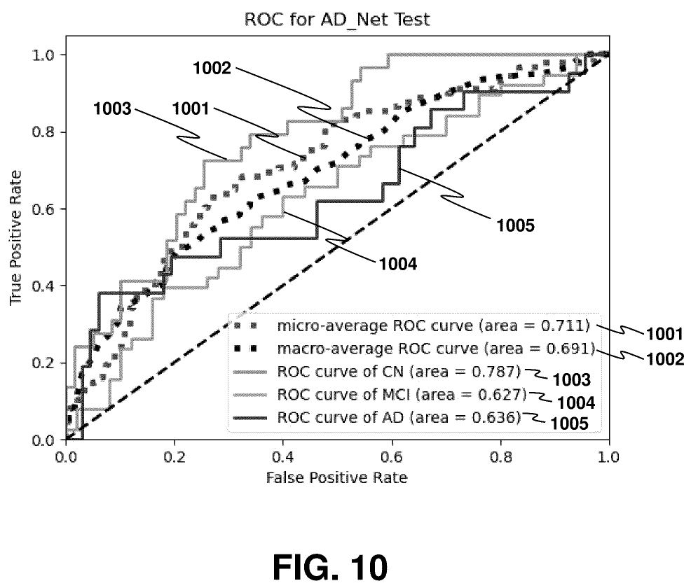

To further explore the performance of our AD_Net model 700, FIG. 10 plots respective ROC curves 1003, 1004, 1005 of the CN, MCI and AD classes along with a micro-average ROC curve 1001 and a macro-average ROC curve 1002. It can be seen from the macro- and micro-average ROC curve areas that the whole AD_Net model 700 has great predictive value.

TABLE I
Cross-validation accuracy of CNN models under test.

| | Performance of 5-fold cross-validation |
|---|---|
| Model | (Mean +/− Standard deviation) |
| Baseline | 0.484 +/− 0.039 |
| AlexNet | 0.434 +/− 0.028 |
| DenseNet | 0.489 +/− 0.056 |
| VGG13 | 0.495 +/− 0.045 |
| VGG16 | 0.509 +/− 0.038 |
| VGG19 | 0.502 +/− 0.034 |
| AD_Net | 0.509 +/− 0.03 |

To be more specific, the AUROC of the MCI condition was lower than each of AUROCs of the CN and AD conditions. It indicates that the MCI condition has a low TPF. The MCI condition remains challenging to classify solely based on MRI images [25]. In contrast, we can see that the CN and AD conditions had good AUROC values, which present decent two-class specificity and modest sensitivity. It means that this model is better at distinguishing between healthy subjects and dementia patients.

### B. Performance of the MLP Model 800

As mentioned above, in the MLP model 800, we divided potential external influencing factors into two groups according to whether they contained directionality and then combined these with VGG16, VGG19, and the plurality of AD_Net feature maps 127 as the input to the MLP model 800 for comparing the predictive performance.

We identified two groups of purely external influences: AGG only and AGMGC only. The names of other corresponding groups are also the names of the CNN model, plus the names of the external influencing factors. As shown in TABLE II, model prediction accuracy is greatly improved after the addition of orientation-influencing factors, viz., the MMSE score and the CDR score.

TABLE II
Cross-validation accuracy of the MLP model 800.

| | Performance of multilayer perceptron |
|---|---|
| Group | (Mean +/− Standard deviation) |
| AGG_Only | 0.486 +/− 0.057 |
| AGMGC_Only | 0.875 +/− 0.033 |
| VGG16 AGG | 0.500 +/− 0.04 |
| VGG16 AGMGC | 0.863 +/− 0.043 |
| VGG19_AGG | 0.489 +/− 0.058 |
| VGG19_AGMGC | 0.877 +/− 0.04 |
| ADNet_AGG | 0.513 +/− 0.025 |
| ADNet_AGMGC | 0.89 +/− 0.018 |

For the control group with AGG influencing factors added, after adding feature maps processed by CNN convolution block, the output accuracy is to some extent improved when VGG16 or VGG19 architecture is used. In contrast, the prediction performance of AD_Net 700 is significantly improved. It not only reduces the standard deviation of the original AD_Net (from 3% to 2.5%), but also improves the accuracy of prediction (from 50.9% to 51.3%).

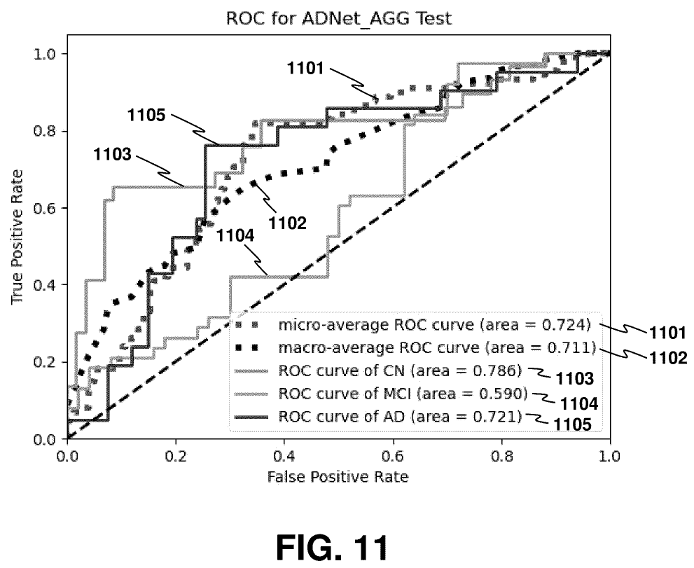

Regarding experimental results obtained for the ADNet AGG model, FIG. 11 plots respective ROC curves 1103, 1104, 1105 of the CN, MCI and AD classes along with a micro-average ROC curve 1101 and a macro-average ROC curve 1102. As can be seen in FIG. 11, the ADNet AGG model was much better than the AD_Net 700 in predicting AD objects. However, the characteristics of two-class specificity and modest sensitivity did not change. There was still no improvement in the AUROC of the MCI condition, and the value of the predictive model remains its ability to distinguish between healthy subjects and dementia patients.

How does the model change when MMSE and CDR are added? When we did not combine the feature images of MRI, the output accuracy of the model is improved by 80%, and the standard deviation was reduced by 42%. It suggests that these two factors can help diagnose AD. After learning the characteristics of these factors, we were able to consider performance when combined with the classic VGG architecture and the AD_Net model 700. After the addition of directional influence factors, the prediction accuracy of all models improved, but the improvement of VGG16 and VGG19 was small and the standard deviation increased.

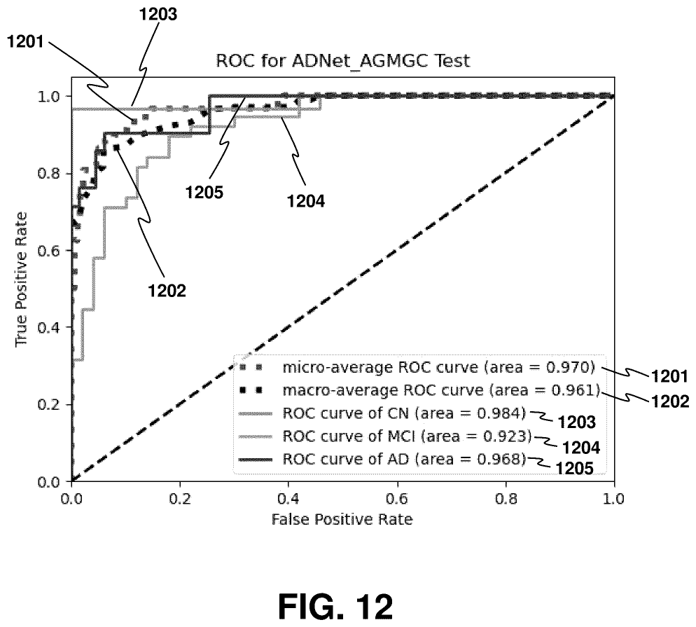

Regarding experimental results obtained for the ADNet AGMGC model, FIG. 12 plots respective ROC curves 1203, 1204, 1205 of the CN, MCI and AD classes along with a micro-average ROC curve 1201 and a macro-average ROC curve 1202. As can be seen from the ROC curves in FIG. 12, after the addition of MMSE and CDR scores, the prediction of AD and CN conditions is improved by more than 20%, and the prediction of the MCI condition is improved by 47.2%. It means that our model compensates for the low predictive ability of MCI. Hence, our model can perform very well when using multifactor diagnosis in real cases.

### IV. Further Evaluation

By the image processing framework 100, a significant advancement is achieved in the multi-class classification of AD, MCI and CN states using MRI data. Our approach is rooted in emerging applications of deep learning in medical imaging, particularly in the context of dementia diagnosis.

Comparison with existing methods: Previous studies have utilized various neural network architectures or are limited to binary AD classification. One significant study employed an attention mechanism using ADNI data but was restricted to differentiating between AD and NC [13]. Another study employed an MLP that combined extracted features with demographic and clinical factors like age, gender, and MMSE scores [26], which is a strategy we have also adopted.

Advancements Over Prior Models: In the realm of multi-class classification, prior studies optimized small 3D CNNs and the VGG13 architecture for classifying the CN, MCI, and AD conditions, achieving accuracies of 61% and 52%, respectively [14], [15]. Both these studies conducted ADNI cross-validation. The present disclosure extends this line of work by employing the VGG19 architecture, known for its deeper network structure compared to VGG13. This choice underpins our model's enhanced robustness and performance.

Performance Analysis: Our model's performance, when evaluated using the ROC diagram, aligns closely with related studies, demonstrating two-class specificity and modest sensitivity [15]. Despite not excelling in identifying AD subjects with the same high performance as some models, our model demonstrates superior capability in distinguishing between CN and MCI subjects. This finding is crucial as early detection of MCI is a critical step in the timely intervention of AD progression. Our study has demonstrated that combining CNN-based models with clinical data to improve the classification of AD stages results in only incremental improvements in predictive accuracy. The significant predictive value of external factors such as age, gender, MMSE score, and CDR score [16] already provides a robust foundation for diagnosis, which may diminish the marginal utility of additional complex CNN models. Despite advanced feature extraction capabilities of these models, the integration of VGG16, VGG19, and the AD_Net model 700 with clinical data did not substantially enhance performance, corroborating findings by [4], which suggest that complex models often offer limited additional benefits over simpler models in well-characterized datasets. Moreover, the integration of CNNs with MLPs, while theoretically advantageous for capturing intricate patterns in data, introduced complexities that resulted in only minor accuracy improvements, as observed in other medical imaging studies [5]. These results highlight the need for a balanced approach to the use of deep learning and traditional clinical predictors within medical diagnostics.

Clinical Implications: The advancements presented in the present disclosure have significant implications for early AD diagnosis. The integration of demographic and clinical data in our MLP model, coupled with the robustness of the VGG19 architecture, suggests a promising avenue for developing more accurate and reliable diagnostic tools in clinical settings. In addition to applying to early diagnosis of AD, the framework 100 can be extended to other similar conditions that can be diagnosed by MRI and the patient's underlying actors.

### V. Embodiments of Present Disclosure

Embodiments of the present disclosure are developed as follows based on the details, examples, applications, etc. regarding the ML-based image processing framework 100 as disclosed above possibly with generalization and extension.

An aspect of the present disclosure is to provide a computer-implemented method for classifying a subject into a CN condition, a MCI condition and an AD condition.

The disclosed method is exemplarily illustrated with the aid of FIGS. 1 and 7. In FIG. 1, an image processing framework 100 in accordance with exemplary embodiments of the present disclosure is shown. The image processing framework 100 as depicted in FIG. 1 illustrates a workflow of processing a raw-image volume 151 of the subject's brain to determine respective likelihoods of the CD, MCI and AD conditions as well as indicates ML models that are employed, viz., an AD_Net model 700 and a MLP model 800. FIG. 7 depicts a realization of the AD_Net model 700 used in the image processing framework 100.

Exemplarily, the disclosed method comprises performing a 3D CNN processing operation 112. The 3D CNN processing operation 112 includes obtaining an image volume 158 of the subject's brain. The image volume 158 provides 3D geometrical details of the brain. The image volume 158 may be prepared from data obtained from 3D imaging of the subject's brain by, for instance, MRI. The 3D CNN processing operation 112 further includes using an AD_Net model 700 to process the image volume 158 to thereby generate a plurality of AD_Net feature maps 127 and a first plurality of scores 128 that respectively predict likelihoods of the CN, MCI and AD conditions. The AD_Net model 700 is an attention-enhanced CNN formed by embedding an attention module 720 into a CNN module 710. The attention module 720 executes an attention mechanism to determine components of relatively high importance in one or more feature maps generated by the CNN module 710. The determined components of relatively high importance are subsequently utilized to assist classification of CN, MCI and AD conditions in a more focused way. The CNN module 710 is a conventional CNN.

The embedding of the attention module 720 into the CNN module 710 to form the AD_Net model 700 is explained in more detail as follows with reference to FIG. 7. The CNN module 710 comprises a plurality of convolutional layers and a plurality of fully connected layers. Pooling, such as max pooling, may be used in the plurality of convolutional layers to downsample or aggregate information in certain intermediate feature maps generated by certain convolution layers in the plurality of convolutional layers. The attention module 720 receives, as input, a first plurality of intermediate feature maps 742 generated by a preselected convolution layer 712, possibly after pooling. The attention module 720 processes the first plurality of intermediate feature maps 742 with an attention mechanism to yield a first plurality of attention-enhanced intermediate feature maps 762. The CNN module 710 continues to process the first plurality of intermediate feature maps 742 to yield a second plurality of intermediate feature maps 746 at a final convolutional layer in the plurality of convolutional layer. Denote a first sequence of computation operations as a sequence of computation operations executed by the CNN module 710 for processing the first plurality of intermediate feature maps 742 to yield the second plurality of intermediate feature maps 746. The first plurality of attention-enhanced intermediate feature maps 762 is processed by a second sequence of computation operations 722 same as the first sequence of computation operations to yield a second plurality of attention-enhanced intermediate feature maps 766. The second plurality of intermediate feature maps 746 (which is not enhanced by the attention mechanism) and the second plurality of attention-enhanced intermediate feature maps 766 are concatenated (by a concatenation operation 716). As a result, the second plurality of intermediate feature maps 746 and the second plurality of attention-enhanced intermediate feature maps 766 are both processed by an initial fully connected layer 714 in the plurality of fully connected layers. It ensures that the plurality of AD_Net feature maps 127 and the first plurality of scores 128, both of which are generated by the plurality of fully connected layers of the CNN module 710, are benefited from utilizing the attention mechanism.

In certain embodiments, the CNN module 710 is realized as a VGG model. The VGG model is a neural network model implemented with a standard deep CNN architecture with multiple layers.

Preferably, the VGG model for realizing the CNN module 710 is a VGG19 model. The VGG19 model has 19 layers with weights, where the 19 layers are formed by 16 convolutional layers and three fully connected layers.

In certain embodiments, the attention module 720 is realized as a CBAM 400.

In certain embodiments, the attention module 720 realized as the CBAM 400 is added to the CNN module 710 realized as the VGG19 model at a location after a final 64-channel convolution layer of the VGG19 model. That is, the aforementioned final 64-channel convolution layer is used as the preselected convolution layer 712.

Note that the AD_Net model 700 is usable to process the image volume 158 only after the AD_Net model 700 is trained. In certain embodiments, the disclosed method further comprises training the AD_Net model 700 before the AD_Net model 700 is used to process the image volume 158. As mentioned above, the ADNI's open-source database [17] may be used to prepare training, validation and test sets used for training the AD_Net model 700.

With the first plurality of scores 128, the subject may be classified into the CN, MCI and AD conditions according to the first plurality of scores 128. However, the first plurality of scores 128 is generated based on the 3D geometrical details of the brain without taking into account other influencing factors of AD. If data of these other influencing factors are available, it is preferable and advantageous to include consideration of these influencing factors in the classification. The resultant classification is generally more accurate than the one based on the 3D geometrical details of the brain alone.

Preferably and advantageously, the disclosed method further comprises performing a MLP prediction operation 113. The MLP prediction operation 113 includes obtaining a plurality of non-directionally influencing factors of AD 135, and a plurality of directionally influencing factors of AD 136. As mentioned above, examples of non-directionally influencing factors of AD 135 include an age, a gender, a GDS score, etc. Similarly, examples of directionally influencing factors of AD 136 include a MMSE score, a CDR score, etc. The MLP prediction operation 113 further includes using a MLP model 800 to process an input to thereby generate a second plurality of scores 138 that respectively predict the likelihoods of the CN, MCI and AD conditions. The input includes the plurality of non-directionally influencing factors of AD 135, the plurality of directionally influencing factors of AD 136, and the plurality of AD_Net feature maps 127. The MLP model 800 is a feedforward artificial neural network formed by a plurality of fully connected layers 810, with a nonlinear activation function 820 used in the plurality of fully connected layers 810 to process outputs of an individual fully connected layer. Furthermore, the MLP model 800 fuses the feature maps with the non-directionally and directionally influencing factors in at least one feature-fusion layer. After the second plurality of scores 138 is obtained, the subject is classified into the CN, MCI and AD conditions according to the second plurality of scores 138.

In certain embodiments, LeakyReLU is used as the activation function 820 in the plurality of fully connected layers 810.

Note that the AD_Net model 700 and MLP model 800 are usable to process the image volume 158 only after the AD_Net model 700 and MLP model 800 are trained. In certain embodiments, the disclosed method further comprises training the AD_Net model 700 and MLP model 800 before the AD_Net model 700 is used to process the image volume 158. As mentioned above, the ADNI's open-source database [17] may be used to prepare training, validation and test sets used for training the AD_Net model 700 and MLP model 800.

Usually, the raw-image volume 151 as directly obtained from brain imaging is not immediately suitable to be processed by the AD_Net model 700. Preprocessing the raw-image volume 151 to obtain the image volume 158 is often required first.

In certain embodiments, the disclosed method further comprises performing a data preprocessing operation 111 before the 3D CNN processing operation 112 is performed. The data preprocessing operation 111 includes obtaining a raw-image volume 151 of the subject's brain, and preprocessing the raw-image volume 151 to generate the image volume 158 such that the image volume 158 is obtained.

In certain embodiments, the raw-image volume 151 is pre-processed by performing at least linear registration 181. Preferably, the raw-image volume 151 is further pre-processed by additionally performing skull removal 182. The raw-image volume 151 may be further pre-processed by additionally performing one or more signal-conditioning operations 183. Examples of the one or more signal-conditioning operations 183 include: bias field correction; normalization and noise cutting; etc.

In certain embodiments, the raw-image volume 151 is pre-processed by performing linear registration 181, skull removal 182, bias field correction, and noise cutting and normalization.

A computing system for classifying a subject into CN, MCI and AD conditions is realizable by including one or more computers, where the one or more computers are configured to execute a process of classifying the subject into the CN, MCI and AD conditions according to any of the embodiments of the disclosed method. An individual computer may be a general-purpose computer, a special-purpose computer such as the one implemented with artificial intelligence processor(s) or graphics processing unit(s), a desktop computer, a physical computing server, a distributed computing server, or a mobile computing device such as a smartphone and a tablet computer.

The present disclosure may be embodied in other specific forms without departing from the spirit or essential characteristics thereof. The present embodiment is therefore to be considered in all respects as illustrative and not restrictive. The scope of the invention is indicated by the appended claims rather than by the foregoing description, and all changes that come within the meaning and range of equivalency of the claims are therefore intended to be embraced therein.

### REFERENCES

There follows a list of references that are occasionally cited in the specification. Each of the disclosures of these references is incorporated by reference herein in its entirety.
[1]D. A. Evans et al., “Prevalence of Alzheimer's Disease in a Community Population of Older Persons: Higher Than Previously Reported,” JAMA, vol. 262, no. 18, pp. 2551-2556, November 1989, doi: 10.1001/jama.1989.03430180093036.
[2]H. Liedes et al., “Multivariate Prediction of Hippocampal Atrophy in Alzheimer's Disease,” J. Alzheimers Dis., vol. 68, no. 4, pp. 1453-1468, January 2019, doi: 10.3233/JAD-180484.
[3]R. Shishegar et al., “Using imputation to provide harmonized longitudinal measures of cognition across AIBL and ADNI,” Sci. Rep., vol. 11, no. 1, Art. no. 1, December 2021, doi: 10.1038/s41598-021-02827-6.
[4]E. E. Bron et al., “Standardized evaluation of algorithms for computer-aided diagnosis of dementia based on structural MRI: The CADDementia challenge,” NeuroImage, vol. 111, pp. 562-579, May 2015, doi: 10.1016/j.neuroimage.2015.01.048.
[5]L. Sørensen et al., “Differential diagnosis of mild cognitive impairment and Alzheimer's disease using structural MRI cortical thickness, hippocampal shape, hippocampal texture, and volumetry,” NeuroImage Clin., vol. 13, pp. 470-482, January 2017, doi: 10.1016/j.nicl.2016.11.025.
[6]Y. Xiao, H. Yin, S.-H. Wang, and Y.-D. Zhang, “TReC: Transferred ResNet and CBAM for Detecting Brain Diseases,” Front. Neuroinformatics, vol. 15, December 2021, doi: 10.3389/fninf.2021.781551.
[7]J. Kim et al., “Development of Random Forest Algorithm Based Prediction Model of Alzheimer's Disease Using Neurodegeneration Pattern,” Psychiatry Investig., vol. 18, no. 1, pp. 69-79, January 2021, doi: 10.30773/pi.2020.0304.
[8]L. Chen, H. Yu, Y. Huang, and H. Jin, “ECG Signal-Enabled Automatic Diagnosis Technology of Heart Failure,” J. Healthc. Eng., vol. 2021, p. e5802722, November 2021, doi: 10.1155/2021/5802722.
[9]J. Fu et al., “Sch-net: a deep learning architecture for automatic detection of schizophrenia,” Biomed. Eng. OnLine, vol. 20, no. 1, p. 75, August 2021, doi: 10.1186/s12938-021-00915-2.
[10]J. Wang, Z. Yu, Z. Luan, J. Ren, Y. Zhao, and G. Yu, “RDAU-Net: Based on a Residual Convolutional Neural Network With DFP and CBAM for Brain Tumor Segmentation,” Front. Oncol., vol. 12, March 2022, doi: 10.3389/fonc.2022.805263.
[11]O. O. Oladimeji and A. O. J. Ibitoye, “Brain tumor classification using ResNet50-convolutional block attention module,” Appl. Comput. Inform., vol. ahead-of-print, no. ahead-of-print, January 2023, doi: 10.1108/ACI-09-2023-0022.
[12]I. D. Apostolopoulos, S. Aznaouridis, and M. Tzani, “An Attention-Based Deep Convolutional Neural Network for Brain Tumor and Disorder Classification and Grading in Magnetic Resonance Imaging,” Information, vol. 14, no. 3, Art. no. 3, March 2023, doi: 10.3390/info14030174.
[13]C. Lian, M. Liu, Y. Pan, and D. Shen, “Attention-Guided Hybrid Network for Dementia Diagnosis With Structural MR Images,” IEEE Trans. Cybern., vol. 52, no. 4, pp. 1992-2003, April 2022, doi: 10.1109/TCYB.2020.3005859.
[14]J. A. Yesavage et al., “Development and validation of a geriatric depression screening scale: a preliminary report,” J. Psychiatr. Res., vol. 17, no. 1, pp. 37-49, 1983 1982, doi: 10.1016/0022-3956(82)90033-4.
[15]A. B. Yoelin and N. W. Saunders, “Score Disparity Between the MMSE and the SLUMS,” Am. J. Alzheimers Dis. Dementias®, vol. 32, no. 5, pp. 282-288, August 2017, doi: 10.1177/1533317517705222.
[16]J. B. Miller and J. S. K. Kauwe, “Predicting Clinical Dementia Rating Using Blood RNA Levels,” Genes, vol. 11, no. 6, Art. no. 6, June 2020, doi: 10.3390/genes11060706.
[17]C. J. Weber et al., “The Worldwide Alzheimer's Disease Neuroimaging Initiative: ADNI-3 updates and global perspectives,” Alzheimers Dement. Transl. Res. Clin. Interv., vol. 7, no. 1, p. e12226, December 2021, doi: 10.1002/trc2.12226.
[18]G. Folego, M. Weiler, R. F. Casseb, R. Pires, and A. Rocha, “Alzheimer's Disease Detection Through Whole-Brain 3D-CNN MRI,” Front. Bioeng. Biotechnol., vol. 8, October 2020, doi: 10.3389/fbioe.2020.534592.
[19]E. H. Corder et al., “Gene dose of apolipoprotein E type 4 allele and the risk of Alzheimer's disease in late onset families,” Science, vol. 261, no. 5123, pp. 921-923, August 1993, doi: 10.1 126/science.8346443.
[20]M. F. Folstein, S. E. Folstein, and P. R. McHugh, “‘Mini-mental state’. A practical method for grading the cognitive state of patients for the clinician,” J. Psychiatr. Res., vol. 12, no. 3, pp. 189-198, November 1975, doi: 10.1016/0022-3956(75)90026-6.
[21]J. C. Morris, “The Clinical Dementia Rating (CDR): current version and scoring rules,” Neurology, vol. 43, no. 11, pp. 2412-2414, November 1993, doi: 10.1212/wn1.43.11.2412-a.
[22]R. Brookmeyer, S. Gray, and C. Kawas, “Projections of Alzheimer's disease in the United States and the public health impact of delaying disease onset.,” Am. J. Public Health, vol. 88, no. 9, pp. 1337-1342, September 1998, doi: 10.2105/AJPH.88.9.1337.
[23]R. N. M. Saleh, M. Hornberger, C. W. Ritchie, and A. M. Minihane, “Hormone replacement therapy is associated with improved cognition and larger brain volumes in at-risk APOE4 women: results from the European Prevention of Alzheimer's Disease (EPAD) cohort,” Alzheimers Res. Ther., vol. 15, no. 1, p. 10, January 2023, doi: 10.1186/s13195-022-01121-5.
[24]J. Zhang, X. Zheng, and Z. Zhao, “A systematic review and meta-analysis on the efficacy outcomes of selective serotonin reuptake inhibitors in depression in Alzheimer's disease,” BMC Neurol., vol. 23, no. 1, p. 210, May 2023, doi: 10.1186/s12883-023-03191-w.
[25]M. S. Albert et al., “The diagnosis of mild cognitive impairment due to Alzheimer's disease: Recommendations from the National Institute on Aging-Alzheimer's Association workgroups on diagnostic guidelines for Alzheimer's disease,” Alzheimers Dement., vol. 7, no. 3, pp. 270-279, 2011, doi: 10.1016/j.jalz.2011.03.008.
[26]S. Qiu et al., “Development and validation of an interpretable deep learning framework for Alzheimer's disease classification,” Brain, vol. 143, no. 6, pp. 1920-1933, June 2020, doi: 10.1093/brain/awaa137.

What is claimed is:

1. A computer-implemented method for classifying a subject into a cognitively normal (CN) condition, a mild cognitive impairment (MCI) condition and an Alzheimer's disease (AD) condition, the method comprising:
obtaining an image volume of the subject's brain; and
using an AD_Net model to process the image volume to thereby generate a plurality of AD_Net feature maps and a first plurality of scores that respectively predict likelihoods of the CN, MCI and AD conditions, wherein the AD_Net model is an attention-enhanced convolutional neural network (CNN) formed by embedding an attention module into a CNN module.

2. The method of claim 1 further comprising:
classifying the subject into the CN, MCI and AD conditions according to the first plurality of scores.

3. The method of claim 1 further comprising:
obtaining a plurality of non-directionally influencing factors of AD, and a plurality of directionally influencing factors of AD;
using a multilayer perceptron (MLP) model to process an input to thereby generate a second plurality of scores that respectively predict the likelihoods of the CN, MCI and AD conditions, wherein the input includes the plurality of non-directionally influencing factors of AD, the plurality of directionally influencing factors of AD, and the plurality of AD_Net feature maps, and wherein the MLP model fuses the feature maps with the non-directionally and directionally influencing factors in at least one feature-fusion layer; and
classifying the subject into the CN, MCI and AD conditions according to the second plurality of scores.

4. The method of claim 3, wherein:
the plurality of non-directionally influencing factors of AD includes one or more first items selected from an age, a gender and a geriatric depression scale (GDS) score; and
the plurality of directionally influencing factors of AD includes one or more second items selected from a Mini-Mental State Examination (MMSE) score and a clinical dementia rating (CDR) score.

5. The method of claim 3, wherein the MLP model comprises a plurality of fully connected layers, and wherein LeakyReLU is used as an activation function in the plurality of fully connected layers.

6. The method of claim 1, wherein the CNN module is realized as a Visual Geometry Group (VGG) model and the attention module is realized as a Convolutional Block Attention Module (CBAM).

7. The method of claim 6, wherein the VGG model is an optimized VGG19 model.

8. The method of claim 7, wherein the CBAM is added to the VGG19 model at a location after a final 64-channel convolution layer of the VGG19 model.

9. The method of claim 1, wherein the image volume is prepared from data obtained from three-dimensionally imaging the subject's brain by magnetic resonance imaging (MRI).

10. The method of claim 1 further comprising:
obtaining a raw-image volume of the subject's brain; and
preprocessing the raw-image volume to generate the image volume such that the image volume is obtained.

11. The method of claim 10, wherein the raw-image volume is pre-processed by performing linear registration, skull removal, bias field correction, and noise cutting and normalization.

12. The method of claim 1 further comprising:
training the AD_Net model before the AD_Net model is used to process the image volume.

13. The method of claim 3 further comprising:
training the AD_Net model and the MLP model before the AD_Net model is used to process the image volume.

14. A computing system for classifying a subject into a cognitively normal (CN) condition, a mild cognitive impairment (MCI) condition and an Alzheimer's disease (AD) condition, the computing system comprising one or more computers configured to execute a process of classifying the subject into the CN, MCI and AD conditions according to the method of claim 1.

15. A computing system for classifying a subject into a cognitively normal (CN) condition, a mild cognitive impairment (MCI) condition and an Alzheimer's disease (AD) condition, the computing system comprising one or more computers configured to execute a process of classifying the subject into the CN, MCI and AD conditions according to the method of claim 2.

16. A computing system for classifying a subject into a cognitively normal (CN) condition, a mild cognitive impairment (MCI) condition and an Alzheimer's disease (AD) condition, the computing system comprising one or more computers configured to execute a process of classifying the subject into the CN, MCI and AD conditions according to the method of claim 3.

17. A computing system for classifying a subject into a cognitively normal (CN) condition, a mild cognitive impairment (MCI) condition and an Alzheimer's disease (AD) condition, the computing system comprising one or more computers configured to execute a process of classifying the subject into the CN, MCI and AD conditions according to the method of claim 4.

18. A computing system for classifying a subject into a cognitively normal (CN) condition, a mild cognitive impairment (MCI) condition and an Alzheimer's disease (AD) condition, the computing system comprising one or more computers configured to execute a process of classifying the subject into the CN, MCI and AD conditions according to the method of claim 5.
